(19) United States
(12) Patent Application Publication
(10) Pub. No.: US 2016/0242950 A1
(43) Pub. Date: Aug. 25, 2016

(54) SHOULDER SUPPORT APPARATUS AND METHODS FOR USING SAME

(71) Applicant: Cory Jess Chase, Pleasant View, UT (US)

(72) Inventor: Cory Jess Chase, Pleasant View, UT (US)

(21) Appl. No.: 14/626,588

(22) Filed: Feb. 19, 2015

(51) Int. Cl. A61F 5/40

(52) U.S. Cl. CPC A61F 5/40; A61F 2220/005

(57) ABSTRACT

The present invention provides an adhesive shoulder support apparatus that is simple in design and which can he easily applied by the user between the shoulder and the adjacent deltoid without requiring the consultation or application of taping to be performed by a physician or physical therapist. The adhesive shoulder support apparatus of the present invention is formed of a stretchable and breathable material that provides sufficient structural tension and support to a shoulder of a user so as to contribute to proper alignment and, accordingly, facilitate a reduction or elimination of shoulder pain caused by core workouts, sports fatigue or injuries, while at the same time promoting healing. A shoulder support apparatus of the present invention comprises a single adhesive member being formed of a durable, resilient material having an adhesive backing. The adhesive support apparatus may be formed including an upper load strap, a lower load strap and a central body portion disposed longitudinally between, said upper and lower load straps and providing a tension load when applied between the shoulder and deltoid of the user so as to simulate an auxiliary muscle. Additionally, the central body portion of the shoulder support apparatus comprises a surface area having a plurality of openings formed therein.

### BACKGROUND

1. The Field of the Invention

This invention relates to a body support apparatus and, in particular, to an adhesive shoulder support apparatus that provides structural support to the shoulder of a user, while providing a load tension sufficient to simulate an auxiliary muscle so as to facilitate a reduction or elimination of shoulder pain and promote healing.

2. The Background Art

The shoulder, a ball-and-socket joint, is made up of three bones, namely, the upper arm bone (humerus), shoulder blade (scapula) and collarbone (clavicle). The shoulder also incorporates four joints, namely, the sternoclavicular joint (SC joint), the acromioclavicular joint (AC joint), the glenohumeral joint (shoulder) and the articulation between the scapula and the ribs. The bones of the shoulder are held together by muscles, tendons and ligaments. Tendons (tough cords of tissue that attach the shoulder muscles to the bone) and ligaments (attach bones the bones) provide additional strength and stability. The rotator cuff is the most important soft tissue structure in the shoulder and essentially holds the “ball” in the “socket” so as to provide mobility and strength to the shoulder joint. Two bursa, located at the highest part of the shoulder, cushion and protect the rotator cuff from the bony arch of acromion and thereby allow smooth movement of the joint.

As to complexity of the shoulder, there are eighteen (18) muscles alone that attach to the scapula. These muscles have to work together at specific ranges of motion to control the scapula on the thoracic spine and subsequently place the shoulder joint in optimal mechanics. As the arm raises overhead, the scapula usually rotates upward 1° for every 2° of humeral motion. If the scapular muscles are not getting the correct neurological input, they generally will not fire at the right time which typically results in scapular dyskinesia. This decrease in scapular control thereby places the glenohumeral joint at a mechanical disadvantage requiring bigger muscles to compensate for smaller ones.

The shoulder is the most movable joint in the body and, accordingly, can be susceptible to instability. To this end, the shoulder may be easily injured by impact or by simple overuse. The shoulder area therefore requires attention to properly strengthen and develop all of the surrounding musculature. Because of the multitude of articulations of the shoulder, a greater risk of trauma exists. As appreciated, an acute shoulder injury can occur suddenly through an impact, strain, sprain or twisting of the shoulder joint. Moreover, with the onset of an injury, pain in the shoulder area may come on for no apparent reason and then gradually become worse over time if not properly treated.

Most people, during their lifetimes, will suffer some type of pain or discomfort in the shoulder region of their body. Some may brush symptoms of shoulder pain aside, attributing their discomfort to a pulled or overstretched muscle. Chronic shoulder issues, however, should not be taken lightly and a proper medical review of any physical ailments or pain associated with the shoulder should be done immediately to diagnose the injury.

Some of the types of shoulder injuries, to name a few, consist of impingement syndrome, bursitis or tendinitis of the muscle, a rotator cuff tear, acromioclavicular joint (AC) separation, deltoid contusion, frozen shoulder, glenoid labrum tear, posterior shoulder dislocation, rotator cuff tendinitis, shoulder sprain, shoulder subluxation, shoulder tendinitis and subscapularis inflammation. In general, each of these conditions means the shoulder is not correctly functioning biomechanically.

One of the most common injuries of the shoulder is a rotator cuff strain, which if left untreated can result in a complete tear. As appreciated, the rotator cuff includes four (4) of the muscles found in the shoulder that are primarily responsible for particular motions or movements of the shoulder. A lot of this motion involves rotation of the humorous, the long bone in the arm, which is why the group of muscles is referred to as rotators. Another major role these muscles play involves the stabilization of the shoulder, which includes a series of joints that requires muscular stabilization to allow for greater range of motion. When one of these muscles, or the tendons that connect the muscles to the bones, becomes torn, either partially or completely, an individual typically has a rotator cuff injury.

Other common injuries of the shoulder may involve dislocation or subluxation which can cause damage to so many structures within the shoulder that the prescribed treatment is widely varied. Structures that can be injured range from the musculature to the ligaments to the labrum and may result in an individual having to undergo surgical intervention.

Many shoulder conditions have similar symptoms and, accordingly, it can often be difficult to diagnose the specific injury or problem from the symptoms alone. A physician, often an orthopedist, will typically diagnose the cause of shoulder pain by taking into consideration the patient's symptoms and medical history, the findings of a physical examination and many times through the aid of diagnostic testing, such as x-rays, a CT scan and/or MRI. The resulting treatment of the shoulder injury and associated discomfort and pain therefore depends on the primary cause of the injury.

Most shoulder injuries may be healed using conservative treatments. However, the healing process typically takes time. The time it takes to recover generally depends upon several factors, such as, for example, the severity of the injury, the type of injury, how quickly one heals and how early the individual begins treatment after the injury. The typical methods of treatment for shoulder pain, for most conditions, involve a combination of rest, exercise, anti-inflammatory medication, applying cold and/or heat to the shoulder joint and in some cases, an injection of steroids into the shoulder joint itself. Doing activities that aggravate shoulder pain often result in further damage, delays in the healing process and may also lead to long term problems requiring surgical intervention. Conversely, not using the shoulder at all tends to weaken the shoulder and may leave it more vulnerable to further injury and discomfort.

Strengthening exercises for the muscles that support the shoulder, particularly the rotator cuff (the muscles and tendons that dynamically stabilized the main shoulder joint) are a major part of treatment for most shoulder injuries. Conversely, strength training before adequate healing has taken place may cause further pain and injury. A physician or physical therapist can usually determine when the shoulder is ready to engage in said strengthening exercises.

As appreciated by those skilled in the art, most shoulder pain improves with undergoing one or more conservative treatments; however, surgery may occasionally be required depending upon the type and severity of the injury. Surgery, which usually involves examination with an arthroscope, may be performed to tighten loose ligaments, repair a damaged or torn tendon, remove a calcium deposit, etc. when conservative treatments are not sufficiently adequately to resolve and improve the underlying symptoms.

One known conservative treatment employed to address shoulder pain and/or discomfort may include the use ofbandages which are strategically applied to particular parts of the body of the patient. For example, when wrapping a shoulder with a bandage (i.e., ACE® Bandage), a first end of the bandage may be wrapped around the deltoid a couple of times to secure its initial positioning relative thereto. The second end of the bandage wrap may then be passed up and over the shoulder joint passing across the chest cavity and under the opposing armpit of the patient. The bandage may then be crossed back over the glenohumeral joint and AC joint and again passed across the chest cavity and under the opposing arm pit, which steps may be performed several times in order to form a “spica” (a Figure-8 configuration with successive V-shaped crossings). The second end of the bandage may terminate either on the shoulder, arm or chest of the patient, wherever desired by the attending physician or physical therapist. The second end of the wrap will usually be secured with hook and loop connections and/or with an elastic taping that retraces the same cross-over “spica” designed application, as demonstrated with the initial wrapping of the bandage. If desired, a white (rigid) tape may then be applied along a portion of the shoulder so as to secure the bandage and the elastic taping in a fixed position. As appreciated, the use of bandage wrapping techniques are generally used by physicians and physical therapists in the art in an effort to isolate and restrain movements of the shoulder, thus effecting a limited range of motion to facilitate healing.

Additionally, a variety of stretchable support tapes (kinesiology tapes) have been developed by those skilled in the art which may be used to aide in the positioning of the shoulder. These stretchable/elastic support tapes generally permit a degree of natural mobility to the shoulder while also acting in a manner to assist in compensating for injured or weak musculature. In particular, the functionality of the kinesiology tape to stretch may facilitate a degree of tension being applied to the supported body part, in effect acting as an auxiliary muscle. Similarly, the use of stretchable support tapes in a correct manner, as prescribed by a physician or physical therapist, are usually intended to provide physical support to the shoulder and thereby increase proprioceptive awareness to the patient.

Significant drawbacks, however, are associated with many of the known techniques and methods that incorporate the use of bandages and kinesiology tapes in treating shoulder injuries. Specifically, the prior art bandages and kinesiology support tapes are commonly sold in either rolls or strips, which then must be customized for the particular application to the shoulder as prescribed by an attending doctor or physical therapist. Attention must also be paid to the particular positioning and placement of the bandages and/or kinesiology tape to effect the desired therapeutic result.

After the desired placement on the patient's body has been carefully determined by the skilled professional, the roll or strips of bandages or elastic tapes must then be cut and properly applied with exactness sufficient to provide the desired pain relief and muscular support to the shoulder. As with most taping methods of the prior art, more than one piece of kinesiology tape is typically used, with particular attention being paid to the percentage of stretch or tension applied in at least a portion of the length of the various pieces of tape comprising the application method, thereby becoming an important factor in the overall process and the resulting therapeutic outcomes.

With the specificity associated with applying prior art wrapping methods and kinesiology taping techniques to achieve a desired shoulder support, pain relief and recovery, a patient must be committed to regular visits to the office of his doctor or physical therapist since the patient has no self-treatment options. Of course, the costs associated with having to engage in frequent doctor visits can be both taxing on the wallet and one's busy schedule.

Furthermore, it may be difficult to be seen by one's doctor on a sufficiently regular basis which seems to be inherently required for these wrapping and taping applications, especially in view of the fact that bandage wrapping and kinesiology taping methods usually equate to a fairly short lifespan.

As appreciated by those skilled in the art, shoulder braces have also been developed and designed to aid in the support and recovery of shoulder injuries. In design, however, shoulder braces of the prior art are typically cumbersome and bulky in nature, including a main body portion that covers at least part of the shoulder and the bicep and, in some embodiments, extends across a portion of the chest cavity. Correspondingly, shoulder braces further include one or more straps that extend from the main body portion over at least a portion of the shoulder, across the chest cavity, under the opposing armpit and across a portion of the patient's back.

Of course, the type of shoulder brace used generally depends upon the injury sustained by the user. For example, some shoulder braces support the clavicle (collarbone) and shoulder area. These types of braces help to keep the collarbone and shoulder area in place allowing a user to move normally while keeping the bones and muscles in the correct position which may ultimately aid in the healing process. Other shoulder braces called shoulder immobilizers are engineered to generally work in keeping the shoulder from moving at all. If a patient has had surgery or a major injury, this is the type of brace that may be useful.

Shoulder braces usually come in defined Levels 1-4 which correlate to the support required, the level of mobility sought and the physical limitations associated with the underlying shoulder injury. Shoulder braces may also be designed in such a manner so as to accommodate cold compression or deep heating wraps for treating pain and inflammation. Although shoulder braces are readily available for purchase in certain retail stores and online, albeit they are considerably more expensive than the alternative of using bandages or kinesiology tape, it is extremely important that the patient know which level of shoulder brace to employ and the proper manner for its use relative to the identification of the specific underlying injury sustained.

Based on the foregoing, what is needed is an adhesive shoulder support apparatus comprised of a stretchable and breathable material that can be easily applied by a user between his/her shoulder and deltoid and which provides sufficient structural tension and support to an injured shoulder so as to facilitate a reduction or elimination of shoulder pain and the promotion of healing. Also what is needed is a one-piece, simple, inexpensive and disposable shoulder support apparatus that does not require consultation or taping application to be performed by a physician or physical therapist, as readily contemplated and required by most prior art methods for wrapping bandages or applying kinesio logy taping techniques.

### SUMMARY OF THE INVENTION

The present invention provides an adhesive shoulder support apparatus that is simple in design and which can be easily applied by the user between his/her shoulder and the adjacent deltoid without requiring the consultation or application of taping to be performed by a physician or physical therapist. The adhesive shoulder support apparatus of the present invention is formed of a stretchable and breathable material that provides sufficient structural tension and support to an injured shoulder of a user so as to contribute to proper alignment and, accordingly, facilitate a reduction or elimination of shoulder pain caused by core workouts, sports fatigue or injuries, while at the same time promoting healing. The shoulder support provides a non-invasive, therapeutic method to effectively promote shoulder stability of a user having sustained a shoulder injury or an athlete when exercising, lifting weights and/or engaging in sporting activities.

The present invention is formed comprising an adhesive backing that exhibits a strong initial bond to the skin when applied and which provides good adhesion with a high tack and yield. The adhesive shoulder support apparatus may be used as a rehabilitation aid for therapies used by physicians, chiropractors and/or physical therapists in an effort to facilitate the reduction and/or elimination of inflammation in the shoulder joints and muscles. The present invention can also be used after a surgical procedure to assist in rehabilitation of a shoulder and may provide a form of structural, yet flexible support, to the shoulder.

The shoulder support apparatus of the present invention is formed as a single adhesive member comprising a central body portion, an upper load strap, a lower load strap, a first extending member and a second extending number. In certain embodiments, the shoulder support apparatus may comprise a longitudinal length that extends from an outer midsection of the upper load strap to an outer midsection of the lower load strap measuring between about 22-28 cm. Correspondingly, the shoulder support apparatus of the present invention may comprise a central width extending between the outer midsection of the first extending member and the outer midsection of the second extending, member which may measure between about 20-25 cm.

In the design of certain embodiments, the adhesive shoulder support apparatus includes a central body portion disposed between the upper load strap and the lower load strap. The central body portion may be formed having a defined surface area comprising both a width and an elongated longitudinal length. Although not required, certain embodiments of the surface area of the central bodyportion of the shoulder support apparatus of the present invention may comprise a plurality of openings formed therein. The function of the openings formed in the central body portion is to aid in providing a form of ventilation and breathability to the underlying, contact area of the shoulder area and deltoid of the user.

One embodiment of the adhesive shoulder support apparatus of the present invention may be selectively applied between an area of the shoulder and along a portion of the longitudinal length of an adjacent deltoid of a user. In design, the adhesive shoulder support apparatus maybe used as a conservative form of treatment wherein being selectively applied to the user in such a manner so as to provide control and maintenance to the positioning of the shoulder joint and, accordingly, alleviate joint pressure and stress on the joints and soft tissue structures in order to provide pain relief, while promoting strength, health and recovery to the shoulder injury. The adhesive shoulder support apparatus, when properly applied between the shoulder and deltoid of the user, may provide enhanced support and in effect act as an auxiliary muscle allowing the shoulder to move freely through a normal range of motion.

In certain embodiments, a method for applying the adhesive shoulder support apparatus of the present invention may comprise the steps of: (1) adhering the upper load strap to the shoulder contiguous the acromion; (2) adhering the central body portion along at least a portion of the longitudinal length of an upper surface area of the deltoid; (3) adhering the lower load strap along a lower portion of the deltoid; and (4) adhering the first and second opposing extending members to the underlying skin of the user in sufficient tension to provide linear stabilization to the respective positioning of the adhesive support apparatus. In an effort to maintain the rotator cuff and the tendons in alignment in order to assist in providing stability to the shoulder, the central body portion of the shoulder support apparatus may have its longitudinal length stretched upon its application between the upper load strap and the lower load strap, thus providing a load tension sufficient to simulate an auxiliary muscle. Due to the flexible characteristics of the shoulder support apparatus, the stretch load or tension applied to the central body portion between the upper load strap and the lower load strap may comprise an extended longitudinal length of between about 0.5-1.5 cm.

The adhesive shoulder support apparatus in accordance with the present invention may be formed of a resilient material having the physical characteristics of being stretchable, breathable and waterproof. In certain embodiments, the resilient material may be comprised of neoprene (wetsuit material) and a highly elastic fabric, such as Lycra®, Spandex® or elastane, being attached to one side. One benefit of the combination of neoprene and a highly elastic fabric is that it provides strength and durable elasticity to the shoulder support apparatus, while being able to be stretched under tension and then return to its original shape. The physical characteristics of the resilient material of the present invention may further provide thermal wannth and breathability to the underlying skin of the user to which the shoulder support apparatus is applied.

In one embodiment, the resilient materials of the present invention comprises an acrylic coated neoprene rubber. The adhesive backing applied to the second side may be moisture resistant, is flexible in nature and can be worn during almost any activity for several days at a time, if desired. Moreover, the composition of the resilient material provides warmth to the area to which it is applied on the user. In certain embodiments, the resilient material comprising the adhesive shoulder support apparatus includes an exterior surface, being. substantially smooth, and an interior surface. The interior surface of the resilient material comprises a surface area that engages an adhesive backing. The adhesive backing may be formed having a first adhesive side that engages the interior surface of the resilient material and a second opposing adhesive side that engages the skin of the user upon application.

When the adhesive shoulder apparatus of the present invention is not applied to the shoulder and deltoid area of a user, the second side of the adhesive backing may include a peel-off backing that can be selectively removed. When applying the adhesive shoulder apparatus of the present invention, the second side of the adhesive backing may be removed in pre-cut sections or as a single piece.

Due to the flexible characteristics of the shoulder support apparatus, the stretch load or tension applied to the central body portion of the shoulder support apparatus between the upper load strap and the lower load strap may comprise an extended longitudinal length of between about 0.5-1.5 cm. In an effort to maintain the rotator cuff and the tendons in alignment in order to assist in providing stability to the shoulder, the central body portion of the shoulder support apparatus may have its longitudinal length stretched upon its application between the upper load strap and the lower load strap, thus providing a load tension sufficient to simulate an auxiliary muscle.

In certain embodiments, the second side of the adhesive backing may be formed to include a plurality of recessed grooves and raised adhesive contact nodules. The recessed grooves and the adhesive contact nodules may be formed in a patterned array or configuration. The respective height of the raised contact nodules are merely sufficient to allow air to flow between the underlying skin of the user and the recessed grooves formed in the second side of the adhesive backing. Correspondingly, the plurality of raised contact nodules are formed and positioned in such a manner so as to provide numerous contact sites between the second side of the adhesive backing and the underlying surface of the skin of the user. In this regard, the second side of the adhesive backing exhibits a strong initial bond producing high tack and peel properties to the adhesive shoulder support apparatus of the present invention.

As appreciated, the adhesive shoulder support apparatus of the present invention may be manufactured in sizes sufficient to extend between a portion of the shoulder and deltoid area of the user. As found in most upper body clothing articles, the shoulder support apparatus may be manufactured in various sizes, including small, medium, large, extra-large and XXL. The adhesive shoulder support apparatus may also be produced in various colors, such as, for example, black, navy, pink, white, green, yellow, red or combinations/variations thereof, just to name a few of the available color options. Furthermore, the shoulder support apparatus of the present invention, if desired, may come with a company design/logo or the name or logo of a college or professional sports team being displayed on the exterior surface of the resilient member.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a top plan view of an embodiment of an adhesive shoulder support apparatus in accordance with the present invention;

FIG. 2 is a bottom plan view of an embodiment of the adhesive shoulder support apparatus in accordance with the present invention;

FIG. 3 is a perspective view of the adhesive shoulder support apparatus selectively applied to a portion of the shoulder and deltoid of a user in accordance with the present invention;

FIG. 4 is a top plan view of a second embodiment of an adhesive shoulder support apparatus in accordance with the present invention;

FIG. 5 is a cross-sectional view of the adhesive shoulder support apparatus in accordance with the present invention;

FIG. 6 is a bottom plan view of the embodiment of the adhesive shoulder support apparatus as shown in FIG. 4; and

FIG. 7 is an exploded view of a second side of an adhesive backing of the adhesive shoulder support apparatus in accordance with the present invention.

### DETAILED DESCRIPTION OF THE INVENTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, but is merely representative of various embodiments of the invention. The illustrated embodiments of the invention will be best understood by reference to the drawings, wherein like parts are designated by like numerals throughout.

The shoulder support apparatus 10 of the present invention is formed as a single adhesive member comprising a central body portion 12, an upper load strap 14, a lower load strap 16, a first extending member 18 and a second extending number 20. In certain embodiments, the shoulder support apparatus 10 may comprise a longitudinal length that extends from an outer midsection of the upper load strap 14 to an outer midsection of the lower load strap 16 measuring between about 22-28 cm. Correspondingly, the shoulder support apparatus 10 of the present invention may comprise a central width extending between the outer midsection of the first extending member 18 and the outer midsection of the second extending member 20 which may measure between about 20-25 cm.

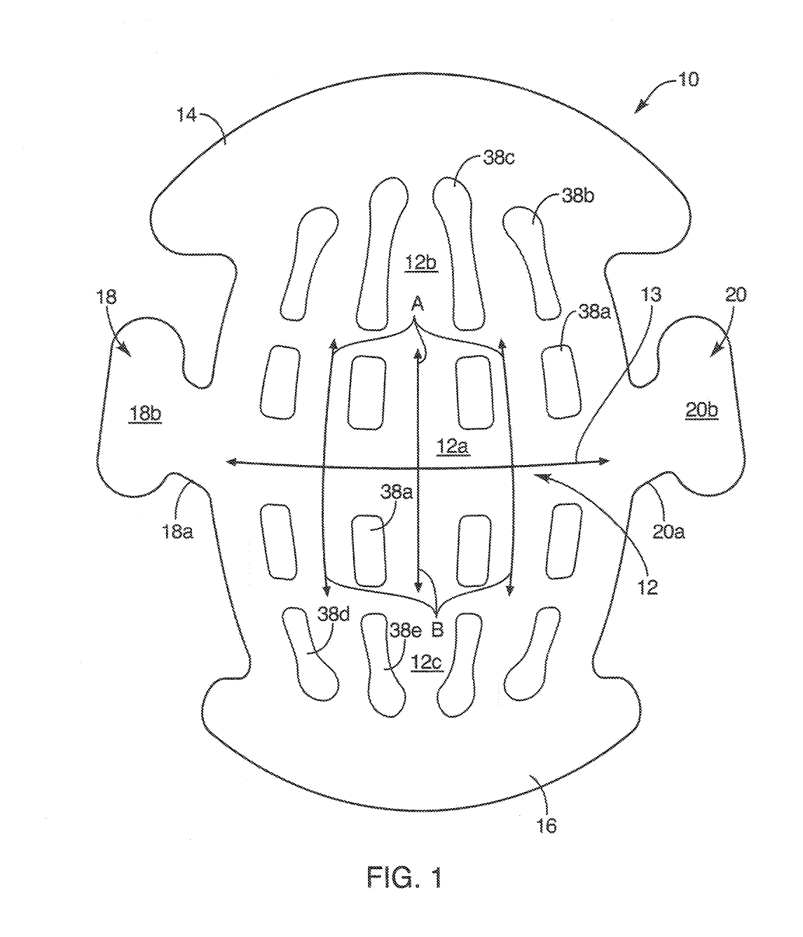

Although the adhesive shoulder support apparatus 10 may be formed as a single, one-piece member, for the purpose of providing a description of its structural and physical characteristics, the shoulder support apparatus 10 may include a central body portion 12 disposed between an upper load strap 14 and a lower load strap 16, as best shown in FIGS. 1-3. The central body portion 12 may be formed having a defined surface area comprising both a width and an elongated length. This surface area of the central body portion 12 may further comprise an upper section 12b, a lower section 12c and a middle section 12a disposed therebetween.

In certain embodiments, the middle section 12a of the surface area of the central body portion 12 may be formed having a width measuring between about 15-17 cm. Where the load strap 14 engages the upper section 12b of the surface area of the central body portion 12, the width of the upper section 12b may be measured at between about 13-15 cm. Correspondingly, at the point where the lower section 12c of the surface area of the central body portion 12 engages the load strap 16, the width of the lower section 12c may be measured at between about 12-14 cm.

The central body portion 12 of the adhesive support apparatus 10 of one embodiment of the present invention may be formed having a middle section 12a comprising a width that is measured to be larger in dimension as compared to tapering at its width measured at the upper section 12b upon engagement with the upper load strap 14 and, correspondingly, at the lower section 12c upon engagement with the lower load strap 16. Additionally, the surface area of the central body portion 12 of the adhesive shoulder support apparatus 10, in certain embodiments, may include a longitudinal length of between about 16-20 cm extending between the upper section 12b and the lower section 12c.

The shoulder support apparatus 10 of the present invention is formed as a single adhesive member comprising a central body portion 12, an upper load strap 14, a lower load strap 16, a first extending member 18 and a second extending number 20. In certain embodiments, the shoulder support apparatus 10 may comprise a longitudinal length that extends from an outer midsection of the upper load strap 14 to an outer midsection of the lower load strap 16 measuring between about 22-28 cm. Correspondingly, the shoulder support apparatus 10 of the present invention may comprise a central width extending between the outer midsection of the first extending member 18 and the outer midsection of the second extending member 20 which may measure between about 20-25 cm.

Although the adhesive shoulder support apparatus 10 may be formed as a single, one-piece member, for the purpose of providing a description of its structural and physical characteristics, the shoulder support apparatus 10 may include a central body portion 12 disposed between an upper load strap 14 and a lower load strap 16, as best shown in FIGS. 1-3. The central body portion 12 may be formed having a defined surface area comprising both a width and an elongated length. This surface area of the central body portion 12 may further comprise an upper section 12b, a lower section 12c and a middle section 12a disposed therebetween.

In certain embodiments, the middle section 12a of the surface area of the central body portion 12 may be formed having a width measuring between about 15-17 cm. Where the load strap 14 engages the upper section 12b of the surface area of the central body portion 12, the width of the upper section 12b may be measured at between about 13-15 cm. Correspondingly, at the point where the lower section 12c of the surface area of the central body portion 12 engages the load strap 16, the width of the lower section 12c may be measured at between about 12-14 cm.

The central body portion 12 of the adhesive support apparatus 10 of one embodiment of the present invention may be formed having a middle section 12a comprising a width that is measured to be larger in dimension as compared to tapering at its width measured at the upper section 12b upon engagement with the upper load strap 14 and, correspondingly, at the lower section 12c upon engagement with the lower load strap 16. Additionally, the surface area of the central body portion 12 of the adhesive shoulder support apparatus 10, in certain embodiments, may include a longitudinal length of between about 16-20 cm extending between the upper section 12b and the lower section 12c.

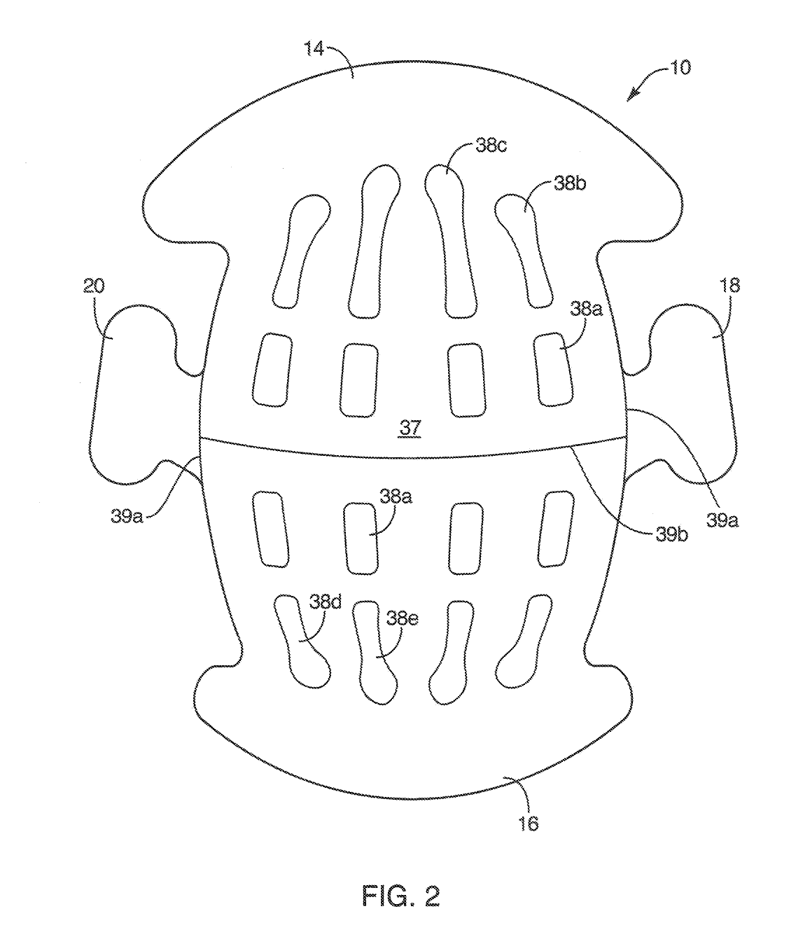

Referring to FIGS. 1-2, the upper load strap 14 may be formed having a central surface area defined by an outer bell-shaped configuration and having opposing rounded ends disposed so as to engage an exterior portion the upper section 12b of the surface area of the central body portion 12 of the shoulder support apparatus 10. In certain embodiments, the width of the upper load strap 14 that extends between the rounded opposing ends may be measured between about 17-20 cm. The longitudinal length between the upper section 12b of the surface area of the central body portion 12 to the corresponding outer peripheral edge of the upper load strap 14 may be measured between about 2.5-5.5 cm.

In certain embodiments of the present invention, the lower load strap 16 may be formed having a central surface area defined by an outer bell-shaped configuration and opposing rounded ends being disposed so as to engage an exterior portion of the lower section 12c of the surface area of the central body portion 12 of the shoulder support apparatus 10. The width of the lower load strap 14 extending between the rounded opposing ends maybe measured between about 14-16 cm. The longitudinal length extending between the lower section 12c of the surface area of the central body portion 12 and the corresponding outer peripheral edge of the lower load strap 16 may be measured between about 2-4 cm.

As best illustrated in FIGS. 1 and 2, the first extending member 18 of the adhesive support member 10 of the present invention may be formed contiguous a portion of the surface area of the middle section 12a of the central body portion 12 and extend outwardly therefrom. In design, the first extending member 18 may comprise a shaft section 18a that protrudes from its engagement with the middle section 12a, wherein the shaft 18a includes opposing parallel sides that extend outwardly and form a rounded head portion 18b attached thereto.

In certain embodiments, the shaft section 18a and the rounded head portion 18b provide a contact surface area of the first extending member 18 for engagement with a portion of the user's body to which the adhesive support member 10 is selectively adhesively applied. In certain embodiments, the shaft section 18a may comprise a longitudinal length measured between about 1-3 cm and a width measured between about 2-4 cm. Correspondingly, the head portion 18b of the first extending member 18 may be formed having a longitudinal length measured from each rounded opposing end between about 4-7 cm and a width measured between about 2-4 cm.

Similar in configuration, the second opposing extending member 20 of the adhesive support member 10 of the present invention may be formed contiguous a portion of the surface area of the middle section 12a of the central body portion 12 and extend outwardly therefrom. In design, the second extending member 20 may comprise a shaft section 20a that protrudes from its engagement with the middle section 12a, wherein the shaft 20a includes opposing parallel sides that extend outwardly and form a rounded head portion 20b attached thereto.

In certain embodiments, the shaft section 20a and the rounded head portion 20b provide a contact surface area of the second extending member 20 for engagement with a portion of the user's body to which the adhesive support member 10 may be selectively applied. The shaft section 20a may comprise a longitudinal length measured between about 1-3 cm and a width measured between about 2-4 cm. Correspondingly, the head portion 20b of the second extending member 20 may be formed having a longitudinal length measured from each rounded opposing end between about 4-7 cm and a width measured between about 2-4 cm.

Although not required, certain embodiments of the middle section 12a of the central body portion 12 of the adhesive shoulder support apparatus 10 of the present invention may comprise a plurality of openings 38 formed therein. The function of the openings 38 formed in the central body portion 12 of the adhesive support apparatus 10 of the present invention is to aid in providing a form of ventilation and breathability to the underlying contact area of the shoulder area and deltoid of the user.

Disposed within the surface area of the middle section 12a of the central body portion 12 are a number of openings 38a. The openings 38a are generally formed in a spaced-apart relationship to each other. In certain embodiments, the openings 38a may comprise a rectangular configuration and be aligned horizontally in a first and second row. The first row may include four (4) openings 38a disposed above the midsection 13 of the central body portion 12 and in spaced-apart relationship to each other and the second row may include four (4) corresponding openings 38a disposed below the midsection 13 and also in spaced-apart relationship to each other.

In certain embodiments, the openings 38a may be formed having a longitudinal length measured between about 1-3 cm and a width measured between about 0.5-1.5 cm. The lower edges of the first row of openings 38a formed in the surface area of the middle section 12a of the central body portion 12 may be disposed between about 2-4 cm from the upper edges of the corresponding openings 38a found in the second row.

Elongated openings 38b-e may also be formed in the central body portion 12 of the shoulder support apparatus 10. The elongated openings 38b of one embodiment of the present invention may be designed having an elongated length that extends between first and second opposing ends, wherein at least a portion of the elongated length comprises a substantially rectangular configuration. The second opposing ends of the elongated openings 38b may be formed having a rounded head portion positioned adjacent the upper load strap 14.

Two (2) elongated openings 38b may be arranged in the portion of the surface area of the upper section 12b of the central body portion 12 in a spaced-apart relationship to contiguous openings 38a formed in the upper row of openings 38a formed in the middle section of the central body portion 12 of the adhesive shoulder support apparatus 10. In one embodiment of the present invention, the elongated openings 38b are disposed in the surface area of the upper section 12b of the central body portion 12 on the sides adjacent the first and second extending members 14, 16, respectively.

In certain embodiments, the elongated openings 38b may be formed having a longitudinal length measured between about 3-5 cm and a width measured between about 0.5-1.5 cm. The lower edges of the elongated openings 38b formed in the surface area of the upper section 12b of the central body portion 12 may be disposed between about 0.5-1.5 cm from the upper edges of the corresponding openings 38a found in the first row of openings formed in the middle section 12a.

The surface area of the upper section 12b of the central body portion 12 may further comprise elongated openings 38c designed with a slightly longer longitudinal length in comparison to the length of the openings 38b. The longitudinal length of the elongated openings 38c extend between first and second opposing ends, wherein at least a portion of the elongated length comprises a substantially rectangular configuration and wherein the respective second opposing ends include a rounded head portion disposed adjacent the upper load strap 14.

Two (2) elongated openings 38c may be arranged in the surface area of the upper section 12b of the central body portion 12 in a spaced-apart relationship to contiguous openings 38a formed in the upper row of openings 38a formed in the middle section 12a of the central body portion 12 of the adhesive shoulder support apparatus 10. In one embodiment of the present invention, the elongated openings 38c are disposed in the surface area of the upper section 12b of the central body portion 12 adjacent each another, while each being disposed adjacent an elongated opening 38b, respectively.

In certain embodiments, the elongated openings 38c may be formed having a longitudinal length measured between about 4-6 cm and a width measured between about 0.5-1.5 cm. The lower peripheral edges of the elongated openings 38c formed in the surface area of the upper section 12b of the central body portion 12 may be disposed between about 0.5-1.5 cm from the upper edges of the corresponding openings 38a found in the first row of openings formed in the middle section 12a.

Additionally, the central body portion 12 may comprise elongated openings 38d designed having a longitudinal length that may be slightly smaller than the length of the elongated openings 38b and larger than the length of the openings 38a. The elongated openings 38d of one embodiment of the present invention may be designed having a longitudinal length that extends between first and second opposing ends, wherein at least a portion of the length comprises a substantially rectangular configuration and wherein the second opposing ends include a rounded head portion disposed adjacent the lower load strap 16.

Two (2) elongated openings 38d may be arranged in the surface area of the lower section 12c of the central body portion 12 of the adhesive shoulder apparatus 10. In one embodiment of the present invention, the elongated openings 38d are disposed in the surface area of the lower section 12c of the central body portion 12 on the sides adjacent the first and second extending members 14, 16, respectively.

In certain embodiments, the elongated openings 38d may be formed having a longitudinal length measured between about 2-4 cm and a width measured between about 0.5-1.5 cm. The upper peripheral edges of the elongated openings 38d formed in the surface area of the lower section 12c of the central body portion 12 may be disposed between about 0.5-1.5 cm from the lower edges of the corresponding openings 38a found in the second row of openings formed in the middle section 12a.

Further, the central body portion 12 may comprise additional elongated openings 38e designed having a longitudinal length that may be slightly larger than the length of the elongated openings 38d and larger than the length of the openings 38a. The elongated openings 38d of one embodiment of the present invention may be designed having a longitudinal length that extends between first and second opposing ends, wherein at least a portion of the length comprises a substantially rectangular configuration and wherein the second opposing ends include a rounded head portion disposed adjacent the lower load strap 16.

Two (2) elongated openings 38e may be arranged in the surface area of the lower section 12c of the central body portion 12 of the adhesive shoulder apparatus 10. In one embodiment of the present invention, the elongated openings 38e are disposed in the surface area of the lower section 12c of the central body portion 12 adjacent each another, while each being disposed adjacent an elongated opening 38d, respectively.

In certain embodiments, the elongated openings 38e may be formed having a longitudinal length measured between about 3-5 cm and a width measured between about 0.5-1.5 cm. The upper peripheral edges of the elongated openings 38e formed in the surface area of the lower section 12c of the central body portion 12 may be disposed between about 0.5-1.5 cm from the lower edges of the corresponding openings 38a found in the second row of openings formed in the middle section 12a.

In certain embodiments, the longitudinal length and width of the elongated openings 38d, 38e, respectively, formed in the lower section 12c of the central body portion 12 of the adhesive shoulder support apparatus 10 may he identical. Accordingly, the dimensional size and shape of the openings 38 that may be formed in the central body portion 12 of the shoulder support apparatus 10 are merely exemplary of certain embodiments of the present invention and are not intended to be restrictive or limiting in construction.

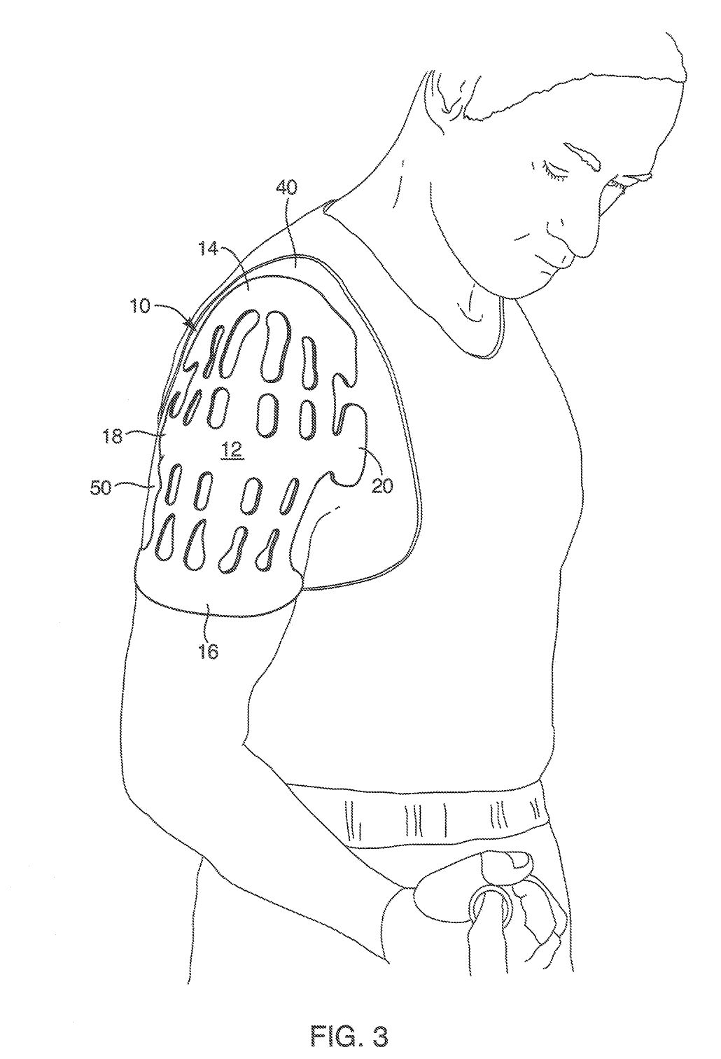

Referring now to FIG. 3, one embodiment of the adhesive shoulder support apparatus 10 of the present invention may be selectively applied between an area of the shoulder 40 and along a portion of the longitudinal length of an adjacent deltoid 50 of a user. In design, the adhesive shoulder support apparatus 10 may be used as a conservative form of treatment wherein being selectively applied to the user in such a manner so as to provide control and maintenance to the positioning of the shoulder joint and, accordingly, alleviate joint pressure and stress on the joints and soft tissue structures in order to provide pain relief, while promoting strength, health and recovery to the shoulder injury. The adhesive shoulder support apparatus 10, when properly applied between the shoulder and deltoid of the user, may provide enhanced support and in effect act as an auxiliary muscle allowing the shoulder to move freely through a normal range of motion.

In certain embodiments, a method for applying the adhesive shoulder support apparatus 10 of the present invention may comprise the steps of: (1) adhering the upper load strap 14 to the injured shoulder 40 contiguous the acromion; (2) adhering the central body portion 12 along at least a portion of the longitudinal length of a upper surface area of the deltoid 50; (3) adhering the lower load strap 16 along a lower portion of the deltoid 50; and (4) adhering the first and second opposing extending members 18, 20 to the underlying skin of the user in sufficient tension to provide linear stabilization to the respective positioning of the adhesive support apparatus 10. In an effort to maintain the rotator cuff and the tendons in alignment in order to assist in providing stability to the shoulder, the central body portion 12 of the shoulder support apparatus 10 may have its longitudinal length stretched upon its application between the upper load strap 14 and the lower load strap 16, along lines AB as shown in FIG. 1, thus providing a load tension sufficient to simulate an auxiliary muscle. Due to the flexible characteristics of the shoulder support apparatus 10, the stretch load or tension applied to the central body portion 12 of the shoulder support apparatus 10 between the upper load strap 14 and the lower load strap 16 may comprise an extended longitudinal length of between about 0.5-1.5 cm.

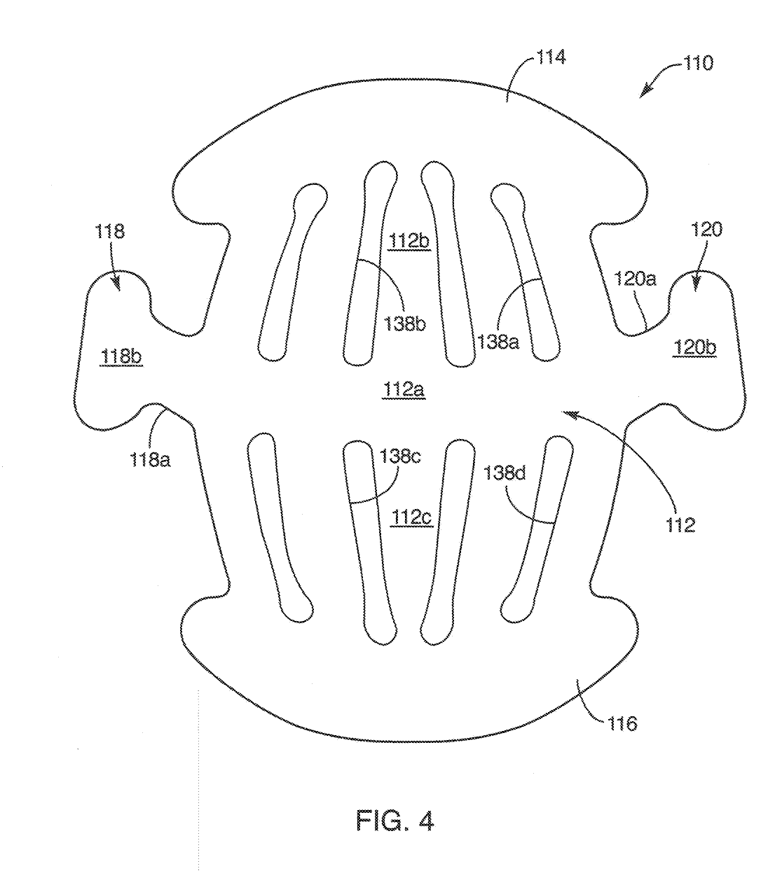

As shown in FIG. 4, another embodiment of the adhesive shoulder support apparatus 110 of the present invention may be formed as a single adhesive member comprising a central body portion 112, an upper load strap 114, a lower load strap 116, a first extending member 118 and a second opposing extending number 120. In certain embodiments, the shoulder support apparatus 110 may comprise a longitudinal length that extends from an outer midsection of the upper load strap 114 to an outer midsection of the lower load strap 116 measuring between about 22-27 cm. Correspondingly, the shoulder support apparatus 110 of the present invention may comprise a central width extending between the outer midsection of the first extending member 118 and the outer midsection of the second extending member 120 which may measure between about 22-27 cm.

Although the adhesive shoulder support apparatus 110 is formed as a single, one-piece member, for the purpose of providing a description of its structural and physical characteristics, the shoulder support apparatus 110 may be formed having a central body portion 112 disposed between an upper load strap 114 and a lower load strap 116. The central body portion 112 may be formed having a defined surface area comprising both a width and a longitudinal length. This surface area of the central body portion 12 may further include an upper section 112b, a lower section 112c and a middle section 112a disposed therebetween.

In certain embodiments, the middle section 112a of the surface area of the central body portion 112 may be formed having a width measuring between about 14-18 cm. Where the load strap 114 engages the upper section 112b of the surface area of the central body portion 112, the width of the upper section 112b may be measured at between about 13-15 cm. Correspondingly, at the point where the lower section 112c of the surface area of the central body portion 112 engages the load strap 116, the width of the lower section 112c may be measured at between about 12-14 cm.

The central body portion 112 of the adhesive support apparatus 110 of one embodiment of the present invention may be formed having a middle section 112a comprising a width that is measured to be larger in dimension as compared to the tapering at its width measured at the upper section 112b upon engagement with the upper load strap 114 and, correspondingly, at the lower section 112c upon engagement with the lower load strap 116. Additionally, the surface area of the central body portion 112 of the adhesive shoulder support apparatus 110, in certain embodiments, may include a longitudinal length of between about 16-20 cm extending between the upper section 112b and the lower section 112c.

Still referring to FIG. 4, the upper load strap 114 may be formed having a central surface area defined by an outer bell-shaped configuration and having opposing rounded ends disposed so as to engage an exterior portion the upper section 112b of the surface area of the central body portion 112 of the shoulder support apparatus 110. In certain embodiments, the width of the upper load strap 114 that extends between the rounded opposing ends may be measured between about 16-20 cm. The longitudinal length between the upper section 112b of the surface area of the central body portion 112 to the corresponding outer peripheral edge of the upper load strap 114 may be measured between about 2.5-5 cm.

In certain embodiments of the present invention, the lower load strap 116 may be formed having a central surface area defined by an outer bell-shaped configuration and opposing rounded ends being disposed so as to engage an exterior portion of the lower section 112c of the surface area of the central body portion 112 of the shoulder support apparatus 110. The width of the lower load strap 116 extending between the rounded opposing ends maybe measured between about 14-16 cm. The longitudinal length extending between the lower section 112c of the surface area of the central body portion 112 and the corresponding outer peripheral edge of the lower load strap 116 may be measured between about 2-5 cm.

The first extending member 118 of the adhesive support member 110 of the present invention may be formed contiguous a portion of the surface area of the middle section 112a of the central body portion 112 and extend outwardly therefrom. In design, the first extending member 118 may comprise a shaft section 118a that protrudes from its engagement with the middle section 112a, wherein the shaft 118a includes opposing sides that extend outwardly and form a rounded head portion 118b attached thereto.

In certain embodiments, the shaft section 118a and the rounded head portion 118b provide a contact surface area of the first extending member 118 for engagement with a portion of the user's body to which the adhesive support member 110 is selectively applied. In certain embodiments, the shaft section 118a may comprise a longitudinal length measured between about 1-3 cm and a width measured between about 2-4 cm. Correspondingly, the head portion 118b of the first extending member 118 may be formed having a longitudinal length measured from each rounded opposing end between about 5-8 cm and a width measured between about 2-4 cm.

Similar in configuration, the second opposing extending member 120 of the adhesive support member 110 of the present invention may be formed contiguous a portion of the surface area of the middle section 112a of the central body portion 112 and extend outwardly therefrom. In design, the second extending member 120 may comprise a shaft section 120a that protrudes from its engagement with the middle section 112a, wherein the shaft 120a includes opposing parallel sides that extend outwardly and form a rounded head portion 120b attached thereto.

In certain embodiments, the shaft section 120a and the rounded head portion 120b provide a contact surface area of the second extending member 120 for engagement with a portion of the user's body to which the adhesive support member 110 may be selectively applied. The shaft section 120a may comprise a longitudinal length measured between about 1-3 cm and a width measured between about 2-4 cm. Correspondingly, the head portion 120b of the second extending member 120 may be formed having a longitudinal length measured from each rounded opposing end between about 5-8 cm and a width measured between about 2-4 cm.

Although not required, certain embodiments of the middle section 112a of the central body portion 112 of the adhesive shoulder support apparatus 110 of the present invention may comprise a plurality of openings 138 formed therein. The function of the openings 138 that may be formed in the central body portion 112 of the adhesive support apparatus 110 of the present invention is to aid in providing a form of ventilation and breathability to the underlying contact area of the shoulder area and deltoid of the user.

Disposed within the surface area of the upper section 112b of the central body portion 112 may be a number of openings 138. The openings 138 are generally formed in a spaced-apart relationship to each other. In certain embodiments, elongated openings 138a-d may be formed in the central body portion 112 of the shoulder support apparatus 110.

The elongated openings 138a of one embodiment of the present invention may be designed having an elongated longitudinal length that extends between first and second opposing ends, wherein at least a portion of the elongated length comprises a substantially rectangular configuration. The second opposing ends of the elongated openings 138a may be formed having a rounded head portion positioned adjacent the upper load strap 114.

In one embodiment of the present invention, the elongated openings 138a are disposed in the surface area of the upper section 112b of the central body portion 112 on the sides adjacent the first and second extending members 114, 116, respectively. Additionally, the elongated openings 138a may be formed having a longitudinal length measured between about 6-8 cm and a width measured between about 0.5-1.5 cm.

The surface area of the upper section 112b of the central body portion 112 may further comprise elongated openings 138b designed with a slightly longer longitudinal length in comparison to the length of the openings 138a. The longitudinal length of the elongated openings 138b extend between first and second opposing ends, wherein at least a portion of the elongated length comprises a substantially rectangular configuration and wherein the respective second opposing ends include a rounded head portion disposed adjacent the upper load strap 114.

In one embodiment of the present invention, the elongated openings 138b may be disposed in the surface area of the upper section 112b of the central body portion 112 adjacent each another, while each being disposed adjacent an elongated opening 138a, respectively. The elongated openings 138b may be formed having a longitudinal length measured between about 6-8 cm and a width measured between about 0.5-1.5 cm.

Additionally, the surface area of the lower section 112c of the central body portion 112 may comprise elongated openings 138c. The longitudinal length of the elongated openings 138c extends between first and second opposing ends, wherein at least a portion of the length comprises a substantially rectangular configuration and wherein the respective second opposing ends include a rounded head portion disposed adjacent the lower load strap 116.

Two (2) elongated openings 138c may be arranged in the surface area of the lower section 112c of the central body portion 112 of the adhesive shoulder apparatus 110. In one embodiment of the present invention, the elongated openings 138c are disposed in the surface area of the lower section 112c of the central body portion 112 on the sides adjacent the first and second extending members 114, 116, respectively.

In certain embodiments, the elongated openings 138c may be formed having a longitudinal length measured between about 6-8 cm and a width measured between about 0.5-1.5 cm. The upper peripheral edges of the elongated openings 138c formed in the surface area of the lower section 112c of the central body portion 112 may be disposed between about 2-4 cm from the lower respective edges of the corresponding openings 138b formed in the upper section 112b.

Further, the central body portion 112 may comprise additional elongated openings 138d designed having a longitudinal length that may be slightly larger than the length of the elongated openings 138c. The elongated openings 138d of one embodiment of the present invention may be designed having a longitudinal length that extends between first and second opposing ends, wherein at least a portion of the length comprises a substantially rectangular configuration and wherein the second opposing ends include a rounded head portion disposed adjacent the lower load strap 116.

Two (2) elongated openings 138d may be arranged in the surface area of the lower section 112c of the central body portion 112 of the adhesive shoulder apparatus 110. In one embodiment of the present invention, the elongated openings 138d are disposed in the surface area of the lower section 112c of the central body portion 112 on the sides adjacent the first and second extending members 114, 116, respectively. Additionally, the elongated openings 138b may be formed having a longitudinal length measured between about 6-8 cm and a width measured between about 0.5-1.5 cm. In certain embodiments, the upper peripheral edges of the elongated openings 138d formed in the surface area of the lower section 112c of the central body portion 112 may be disposed between about 2-4 cm from the lower edges of the corresponding openings 138a formed in the upper section 112b.

In certain embodiments, the longitudinal length and width of the elongated openings 138c, 138d, respectively, formed in the lower section 112c of the central body portion 112 of the adhesive shoulder support apparatus 110 may be identical. Accordingly, the dimensional size and shape of the openings 138 that may be formed in the central body portion 112 of the shoulder support apparatus 110 are merely exemplary of certain embodiments of the present invention and are not intended to be restrictive or limiting in construction.

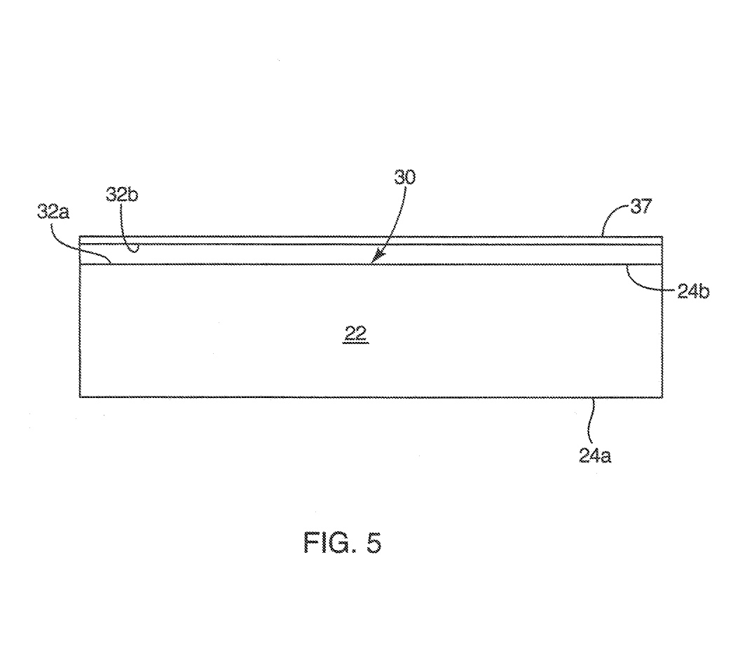

As best illustrated in FIG. 5, the adhesive shoulder support apparatus 10 in accordance with the present invention may be formed of a resilient material 22 having the physical characteristics of being stretchable, breathable and waterproof In certain embodiments, the resilient material 22 may be comprised of neoprene (wetsuit material) and a highly elastic fabric, such as Lycra®, Spandex® or elastane, attached to one side. One benefit of the combination of neoprene and highly elastic fabrics or synthetic fibers is that it provides strength and durable elasticity to the shoulder support apparatus 10, while being able to be stretched under tension and then return to its original shape. The physical characteristics of the resilient material 22 of the present invention may further provide thermal warmth and breathability to the underlying skin of the user to which the shoulder support apparatus 10 is applied. In one embodiment, the resilient material 22 may be formed having a thickness of between about 58-60 mils together with a medical grade acrylic pressure sensitive adhesive backing 30 having a thickness of between about 1.5-3 mils. **It is appreciated that the relative thickness of the resilient material 22 and its adhesive backing 30, respectively, as provided herein is not intended to be a limitation or dimensional restriction, but rather as exemplary of one embodiment of the present invention of the adhesive shoulder support apparatus 10.

In certain embodiments, the resilient material 22 comprising the adhesive shoulder support apparatus 10 includes an exterior surface 24a, being substantially smooth, and an interior surface 24b. The interior surface 24b of the resilient material 22 comprises a surface area that engages an adhesive backing 30. The adhesive backing 30 may be formed having a first adhesive side 32a that engages the interior surface 24b of the resilient material 22 and a second opposing adhesive side 32b.

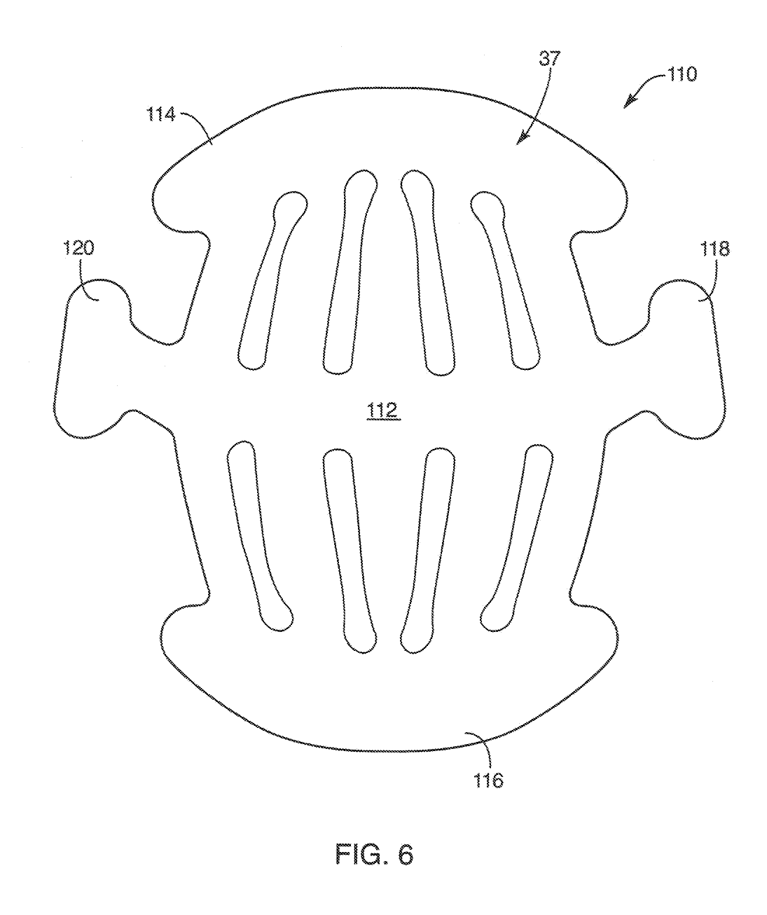

When the adhesive shoulder apparatus 10 of the present invention is not applied to the shoulder and deltoid area of a user, the second side 32b of the adhesive backing 30 may include a peel-off backing 37 that can be selectively removed. When applying the adhesive shoulder apparatus 10 of the present invention, the second side 32b of the adhesive backing 30 may be removed in pre-cut sections, as defined by the pre-cuts 39a, 39b, as illustrated in FIG. 2 or as a single piece as illustrated in FIG. 6.

With reference to FIG. 2, the defined pre-cuts 39a, 39b that may be formed in the peel-off backing 37 of one embodiment of the present invention produce four (4) sectional pieces that can each be removed independent of the other. According to certain methods for applying the adhesive shoulder support apparatus 10 to the desired shoulder area of the user, the sectional piece of the peel-off backing 37 that selectively covers the second side 32b of the adhesive backing 30 extending between the midsection 13 of the central body portion 12 and the upper load strap 14 may be removed and an upper portion thereof applied to an area of the shoulder 40 contiguous the acromion, as best shown in FIG. 3. The sectional piece of the peel-off backing 37 that selectively covers the second side 32b of the adhesive backing 30 extending between the midsection 13 of the central body portion 12 and the lower load strap 16 may then be removed and applied from the point of contact at the area of the shoulder 40 with a stretch load or tension applied to the central body portion 12 of the shoulder support apparatus 10 that extends down to the application of the lower load strap 16 to the portion of surface area of the deltoid 50.

Due to the flexible characteristics of the shoulder support apparatus 10, the stretch load or tension applied to the central body portion 12 of the shoulder support apparatus 10 between the upper load strap 14 and the lower load strap 16 may comprise an extended longitudinal length of between about 0.5-1.5 cm. In an effort to maintain the rotator cuff and the tendons in alignment in order to assist in providing stability to the shoulder, the central body portion 12 of the shoulder support apparatus 10 may have its longitudinal length stretched upon its application between the upper load strap 14 and the lower load strap 16, along. lines AB as shown in FIG. 1, thus providing a load tension sufficient to simulate an auxiliary muscle. Additionally, the pre-cut sectional pieces of the pre-cut backing 37 which selectively covers the second side 32b of the adhesive backing 30 that extends over the first and second opposing extending members 18, 20 may be removed so that the second adhesive side 32b can be applied to the underlying skin of the user in sufficient tension to provide linear stabilization to the respective positioning of the adhesive support apparatus 10.

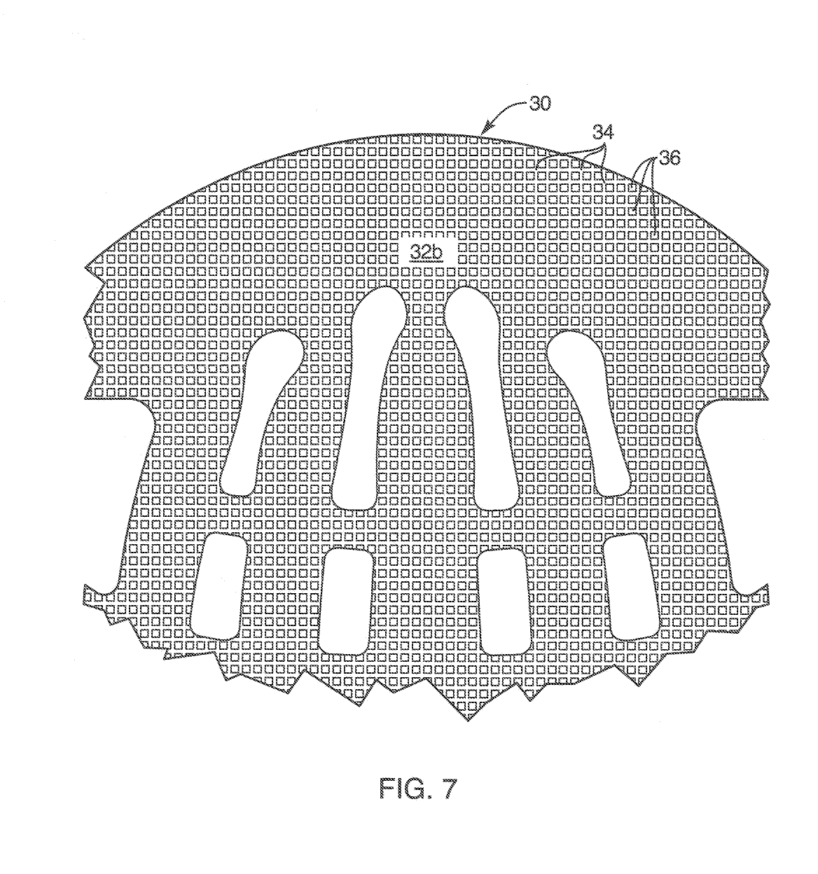

Turning now to FIG. 7, the second side 32b of the adhesive backing 30 may be formed to include a plurality of recessed grooves 34 and raised adhesive contact nodules 36. In certain embodiments of the present invention, the recessed grooves 34 and the adhesive contact nodules 36 may be formed in a patterned array or configuration. Functionally, the patterned array or configuration of the recessed grooves 34 and the raised contact nodules 36 of the second side 32b may provide elasticity to the adhesive backing 30 sufficient to accommodate the stretchability of the central body portion 12 of the adhesive shoulder support apparatus 10. The respective height of the raised contact nodules 36 may be sufficient to allow air to flow between the underlying skin of the user and the recessed grooves 34 formed in the second side 32b of the adhesive backing 30. Correspondingly, the plurality of raised contact nodules 36 are formed and positioned in such a manner so as to provide numerous contact sites between the second side 32b of the adhesive backing 30 and the underlying surface of the skin of the user. In this regard, the second side 32b of the adhesive backing 30 exhibits a strong initial bond producing high tack and peel properties to the adhesive shoulder support apparatus of the present invention.

As appreciated, the adhesive shoulder support apparatus 10, 110 may be manufactured in sizes sufficient to extend between a portion of the shoulder and deltoid area of the user. As found in most upper body clothing articles, the shoulder support apparatus 10, 100 may be manufactured in various sizes, including small, medium, large, extra-large and XXL. It is further appreciated that the measurements provided herein are merely exemplary of certain embodiments of the present invention and are not intended to be interpreted as limiting, in relation to the overall dimensions of the adhesive shoulder support apparatus 10, 110. Additionally, the adhesive shoulder support apparatus 10, 110 may be produced in various colors, such as, for example, black, navy, pink, white, green, yellow, red or combinations/variations thereof, just to name a few of the available color options. Furthermore, the shoulder support apparatus 10, 110 of the present invention, if desired, may come with a company design/logo or the name or logo of a college or professional sports team being displayed on the exterior surface 24a of the resilient member 22.

The present invention may be embodied in other specific forms without departing from its fundamental functions or essential characteristics. The described embodiments are to be considered in all respects only as illustrative, and not restrictive. All changes which come within the meaning and range of equivalency of the illustrative embodiments are to be embraced within their scope.

What is claimed and desired to be secured by United States Letters Patent is:

1. A shoulder support apparatus, comprising:
a single adhesive member being formed of a durable, resilient material having an adhesive backing;
said adhesive member comprising an upper load strap, a lower load strap, and a central body portion disposed longitudinally between said upper and lower load straps; and
said central body portion compris ng a surface area having a plurality of openings formed therein.

2. The shoulder support apparatus as defined in claim 1, wherein said resilient material comprises neoprene in combination with a highly elastic fabric.

3. The shoulder support apparatus as defined in claim 1, wherein said adhesive backing comprises a plurality of recessed grooves and raised adhesive contact nodules.

4. The shoulder support apparatus as defined in claim 1, wherein said longitudinal length of said adhesive member extending between. said upper load strap and said lower load strap comprises between about 22-28 cm.

5. The shoulder support apparatus as defined in claim 1, wherein said adhesive member further comprises first and second extending members that extend outwardly from said central body portion on opposing sides thereof.

6. The shoulder support apparatus as defined in claim 5, wherein said adhesive member comprises a width extending between said first and second extending members, said width measured between about 20-28 cm.

7. The shoulder support apparatus as defined in claim 5, wherein said first and second extending members comprise a contact surface area including an extending shaft section having opposing parallel sides that extend outwardly from said central body to form a rounded head portion.

8. The shoulder support apparatus as defined in claim 1, wherein said surface area of said central body portion comprises a longitudinal length measured between about 16-20 cm.

9. The shoulder support apparatus as defined in claim 1, wherein said surface area of said central body portion comprises a width measured between about 12-17 cm.

10. The shoulder support apparatus as defined in claim 1, wherein said upper load strap comprises a central surface area defined by an outer bell-shaped configuration and opposing rounded ends.

11. The shoulder support apparatus as defined in claim 10, wherein said central surface area of said upper load strap comprises a width measured between about 17-20 cm.

12. The shoulder support apparatus as defined in claim 10, wherein said central surface area of said upper load strap comprises a longitudinal length measured between about 2.5-5.5 cm.

13. The shoulder support apparatus as defined in claim 1, wherein said lower load strap comprises a central surface area defined by an outer bell-shaped configuration and opposing rounded ends.

14. The shoulder support apparatus as defined in claim 13, wherein said central surface area of said lower load strap comprises a width measured between about 14-46 cm.

15. The shoulder support apparatus as defined in claim 13, wherein said central surface area of said lower load strap comprises a longitudinal length measured between about 2-4 cm.

16. A method for applying a shoulder support apparatus, comprising the steps of:
obtaining a single adhesive member formed of a durable, resilient material having an adhesive backing, wherein said adhesive member comprises:
an upper load strap;
a lower load strap;
a central body portion disposed longitudinally between said upper and lower load straps and comprising a surface area having a length and width;
first and second extending members projecting outward on opposing sides of said width of said central body portion; and
wherein said suiface area of said central body portion comprises a plurality of openings formed therein;
adhering the adhesive backing of the upper load strap of the single adhesive member to a shoulder area of a user;
adhering the adhesive backing of the central body portion along at least a portion of a longitudinal length of an upper surface area of an adjacent deltoid of said user;
applying a stretch to said length of said central body portion upon application to produce a load tension between said shoulder and deltoid of said user sufficient to simulate an auxiliary muscle;
adhering the adhesive backing of the lower load strap of the single adhesive member to a lower portion of said deltoid; and
adhering the adhesive backing of the first and second opposing extending members to adjacent underlying skin of said user in sufficient tension to provide linear stabilization to said single adhesive member.

17. The method for applying a shoulder support apparatus as defined in claim 16, further comprising the step of maintaining the rotator cuff and tendons of said user in alignment to assist with stability of said shoulder.

18. The method for applying a shoulder support apparatus as defined in claim 16, further comprising the step of providing control and maintenance to proper positioning of said shoulder.

19. The method for applying a shoulder support apparatus as defined in claim 16, further comprising the step of alleviating joint pressure and stress on joints and soft tissue structures of said shoulder to facilitate pain relief and promote strength and recovery.

20. The method for applying a shoulder support apparatus as defined in claim 16, wherein said resilient material comprises neoprene in combinationwith a highly elastic fabric.
